SEQUENCE LISTING (5 sequences)

SEQ ID NO: 1 - protein, 39 aa, Rift Valley fever virus
  PEPTIDE (1)..(39): RVFV ANTIGEN
SAHYLNNDGKMASVKCPPKYELTEDCNFCRQMTGASLKK

SEQ ID NO: 2 - DNA, 23 bp, Artificial Sequence
  Activation-Induced (Cytidine) Deaminase Forward PCR Primer
cgtggtgaagaggagagatagtg

SEQ ID NO: 3 - DNA, 23 bp, Artificial Sequence
  Activation-Induced (Cytidine) Deaminase Reverse PCR Primer
cagtctgagatgtagcgtaggaa

SEQ ID NO: 4 - DNA, 20 bp, Artificial Sequence
  Glyceraldehyde 3-Phosphate Dehydrogenase Forward PCR Primer
tgtgtccgtcgtggatctga

SEQ ID NO: 5 - DNA, 22 bp, Artificial Sequence
  Glyceraldehyde 3-Phosphate Dehydrogenase Reverse PCR Primer
cctgcttcaccaccttcttgat

(19) United States
(12) Patent Application Publication
(10) Pub. No.: US 2016/0046907 A1
(43) Pub. Date: Feb. 18, 2016

(54) IN VITRO SYSTEM FOR GENERATION OF ANTIGEN-SPECIFIC IMMUNE RESPONSES

(71) Applicant: SOUTH DAKOTA STATE UNIVERSITY, Brookings, SD (US)

(72) Inventor: Alan John Young, Brookings, SD (US)

(73) Assignee: SOUTH DAKOTA STATE UNIVERSITY, Brookings, SD (US)

(21) Appl. No.: 14/613,354

(22) Filed: Feb. 3, 2015

Related U.S. Application Data
(60) Provisional application No. 61/935,321, filed on Feb. 3, 2014.

(51) Int. Cl. C12N 5/0781; C07K 16/28; G01N 33/68

(52) U.S. Cl. CPC C12N 5/0635; G01N 33/6854; G01N 33/6872; C07K 16/28; C12N 2502/1121; C12N 2502/11; C12N 2506/11; C12N 2501/998; G01N 2333/70503; G01N 2333/70553; C07K 2317/14; C07K 2317/75

(57) ABSTRACT

The present invention describes methods to produce vaccines and antibodies, which methods including contacting follicular dendritic cells (FDC) with naïve B-cells to mimic conditions in the germinal center (CG) in vitro, including methods of enhancing antibody production in hybridoma cells and compositions comprising product of the instant methods.

### CROSS REFERENCE TO RELATED APPLICATION

This application claims benefit under 35 U.S.C. §119(e) to U.S. Provisional Application No. 61/935,321, filed Feb. 3, 2014, which is incorporated by reference herein in its entirety.

This application was made with government support under Grant No. 1R15A1072757-01 awarded by the National Institutes of Health (NIH). The government has certain rights in the invention

### BACKGROUND OF THE INVENTION

1. Field of the Invention

The present invention relates generally to development of immunological agents, and more specifically to an in vitro method of producing high affinity antibodies.

2. Background Information

One of the main functions of the immune system is to produce high affinity antibody secreting plasma cells and memory B cells. To accomplish this, direct contact between activated B cells and T cells is necessary. There are specialized microenvironments, termed germinal centers (GCs) that are formed as a consequence of the interaction of B and T cells. The GC produces high affinity antibody-secreting plasma cells and memory B cells that provide long-lived protective humoral immunity.

The key regulatory cell in the GC is the follicular dendritic cell (FDC). The exact origin of FDCs is not fully understood. However, it is believed that FDCs either develop by additional differentiation of a subset of follicular stromal cells of mesenchymal origin or FDCs develop from precursor cells. A key function of FDCs is to present antigen, in the form of immune complexes, to both naïve B cells in primary follicles to activate them, and to antigen printed GC B cells to increase their maturation affinity. Naïve B cells can transport immune complexes from the subcapsular sinus of lymph nodes to FDCs. FDCs present immune complexes to newly activated B cells which compete for antigen during the progression of antibody affinity maturation. Several studies have established that FDCs present immune complexes that stimulate antigen activated B cells, inducing the expression of activation induced cytidine deaminase (AID). This process leads to somatic hypermutation and class switch recombination.

GCs are temporary structures and their organization is temporally regulated. The development of GCs can be divided into various steps. GCs are first detectable four days post-immunization. A GC can be subdivided into two zones; the light zone is where non-dividing GC B cells (centrocytes) live within the elaborate FDC network, and the dark zone is where GC B cells live outside the FDC network. FDCs are most dense in the light zone of the GC, although some FDCs can be found in the dark zone. Rapid proliferation of activated GC B cells (centroblasts) occurs in the dark zone. This is also when GC B cells' variable genes are modified by somatic hypermutation mechanisms. Within the light zone, GC B cells compete for survival signals based on the affinity of their B cell receptor (BCR) to the presented antigen. The non-proliferating centrocytes are scattered among FDC processes and T-follicular helper (TFH) cells for selection and affinity maturation events. Fluorescent and in vivo imaging have characterized the importance of the dark and light zones. Proliferation occurs primarily within the dark zone and B cells predominately migrate from dark zone to light zone, although cells can travel from the light zone back to the dark zone.

Costimulatory antigen-specific, interactions between follicular B cells and T helper cells occurs in T cell zones of secondary lymphoid organs to generate GC precursor B cells. This process triggers the formation of GCs within the B cell zone of secondary lymphoid organs, including the spleen and lymph nodes. Some of the GC precursor B cells travel into adjacent B cell follicles and promote GC formation by immense proliferation within the stromal environment generated by FDCs. GC reactions develop in the follicles of secondary lymphoid organs in response to T cell-dependent antigens. GCs are made up of a T cell zone and B cell zone. Primary follicles of naïve B cells or secondary follicles of activated B cells are referred to as the B cell zone within the GC. B cells can become either plasma cells or memory B cells each is long-lived and protects the host against subsequent exposure to the same antigen or to help clear persistent primary infections. B cells circulate in the blood, enter secondary lymphoid organs, and eventually travel through the B cell follicle in search of a matching antigen. The B cell receptor (BCR) is responsible for the recognition of foreign antigen by a B cell. BCRs can freely bind small antigens; however, larger antigens require presentation by specialized antigen presenting cells (APCs) capable of displaying unprocessed antigen on their cell surface. B cells filter through the follicle for approximately 24 hours and, if unable to find their cognate antigen, will re-enter the blood stream and migrate to other secondary lymphoid structures.

There are varying ways an antigen can enter into the B cell follicle, depending on the molecular weight of the antigen. Multi-photon intravital imaging of lymph nodes has visualized that antigen is initially delivered into the subcapsular sinus (SCS) by afferent lymphatics and is transferred across the SCS lining by specialized macrophages. Proximal to the outer B cell follicle, closest to the SCS, the lining of specialized macrophages spreads out into both the SCS and B cell follicle and inhibits the free diffusion of antigen into the inner lymphocyte cortex. For small antigens up to 70 kilodaltons, there are follicular channels formed by collagen-rich fibers and fibroblastic reticular cells. These channels spread from the SCS to the FDCs within the B cell follicle. Particulate or opsonized antigens are captured by SCS macrophages and transported along the cell membranes to the B cell follicle for presentation to B cells.

High affinity antibodies are produced through clonal expansion of antigen-activated B cells. To become activated, B cells must encounter antigen; that usually requires a focused effort from other immune cell subsets including macrophages, dendritic cells, and T cells. B cells that recognize antigen are activated and begin to repolarize to the T-B border in search of T cell helper activation. The corresponding antigen-activated T cell then generates the secondary signals needed for GC B cells or extra-follicular plasma cell differentiation. After T cell stimulation, activated B cells travel back to the outer-follicular regions for clonal expansion before the GC formation is initiated. During the first 3 days after immunization, antigen activated B cells present antigen and repeatedly form conjugates with antigen-specific CD4⁺ T cells at the T-B border. The length of time of interaction between T and B cells is critical for forming GC B cells. Surface bound molecules and secreted cytokines also play important roles in the early differentiation of GC B cells, during T and B cell interactions. Pre-GC B cells then move to a central area of the B cell follicle and begin their rapid proliferation that creates GCs within the FDC network.

During GC B cell proliferation, somatic hypermutation of Ig variable heavy chains and isotype switching from IgM to other isotypes occurs. Point mutations are introduced by somatic hypermutation as mutagenic repair mechanisms of induced U:G mismatch pairings in the Ig variable region, creating diversification of Ig specificity. For B cells to undergo somatic hypermutation, antigen must be present and the amount of somatic hypermutation increases if the antigen is presented in the firm of an immune complex.

Affinity maturation occurs next through B cell replication with associated mutation and selection. Since B cells have a 10⁻³ mutation rate compared to the 10⁻⁸ mutation rate in other cells, each replication of as B cell will result in somatic mutation. The ability of these newly replicated GC B cells with somatic hypermutation is dependent on their capacity to bind antigen; those that bind antigen at higher affinity are preferentially chosen fin survival and differentiation. While not being bound by theory, a model of affinity maturation indicates that newly formed GC B cells with higher affinity BCRs are selected because they capture antigen from FDCs more effectively and are able to present peptides derived from these antigens to comparatively low affinity matching TFH cells. Affinity maturation requires protein stability, lack of autoimmunity and high affinity of antibodies to entering antigens. The balancing of these attributes is attained only when antibody producing B cells heighten their mutation rates (somatic hypermutation) to a level that is not too low to find potency increasing mutations but not too high to destroy stable immunoglobulins and/or existing affinity.

FDCs appear to play a critical role in saving GC B cells from apoptosis. GC B cells that do not effectively bind or receive adequate T cell help will undergo apoptosis with the apoptotic bodies being removed by GC macrophages. GC B cells with high affinity are positively selected and can differentiate into either antibody secreting plasma cells or memory B cells that can exit the GC through the adjoining mantle zone. In vitro studies have indicated that isolated GC B cells deteriorate rapidly by apoptosis upon culture at 37° C.

Physical contact between B cells and FDCs is required for B cell survival during culture. The important role that FDC contact plays in rescuing B cells from apoptosis is apparent after observing that clustering of FDCs and B cells occurs rapidly and the clustered B cells survive while the B cells not included in the cluster die as a result of apoptosis. The addition of FDCs to B cell cultures decreases the number of apoptotic cells. When FDCs were present during cultures, only a small percentage of the non-clustered B cells had an apoptotic appearance after 24 or 72 hours, indicating that the presence of FDCs in B cell cultures is adequate in preventing apoptosis of the B cells. Following culture, about 30% of the B cells were found inside clusters associated with an FDC and only the clustered B cells were viable. B cells become completely enveloped by FDCs during culture.

After selection, GC B cells go through class switch recombination which is a process that irreversibly rearranges the immunoglobulin (Ig) heavy chain constant region genes from IgM or IgD to IgG, IgA or IgE. Class switch recombination allows effector function to change while maintaining antigenic specificity. AID controls class switch recombination, which B cells can undergo as early as the first reception of T cell help at the T-B border where AID is quickly unregulated. Proliferating centroblasts of the GC dark zone also need AID to undergo somatic hypermination. B cells are then allowed to differentiate into either long-lived plasma cells or memory B cells or they can endure more rounds of somatic hypermutation,

Generation of long-lived, high affinity antibody responses underlies the success of most vaccines, is vital for the survival of neonates exposed to life-threatening infections, and protects the host from re-infection. The best way to achieve robust, lasting immunity to infection relies on the survival of the host from natural infection; however morbidity and mortality are obviously key concerns. Strategies have been developed to help either boost the natural immune system against foreign antigens, or to treat disease once the host is infected.

One of the options in the treatment of diseases is the administration of intravenous immunoglobulin (IVIG). Unfortunately, the amount of specific immunoglobulin for this therapy is a restricting factor. IVIG produced in animals can be used in humans, however more than one dose of any non-human antibody triggers what is known as sentin-sickness: a natural response to species-specific epitopes that generates a neutralizing memory response against the foreign antibodies and prevents subsequent use. Several alternatives have been investigated to overcome this obstacle. Serum antibody can be obtained from human donors who have already been exposed to the infectious antigen. However, there is the risk of contamination with human pathogens such as hepatitis C virus, hepatitis B virus and HIV. The quantity of this human serum antibody is limited and ethical considerations impede the active production of specific antisera by immunization. While fully human antibodies have been generated by use of vaccinating transgenic animals, generating transgenic animals is complicated, arduous and has failed to produce commercial levels of antibody.

Technology is needed that removes the dependence on in vivo hosts for the production of polyclonal and monoclonal antibodies, which technology mimics the natural GC reaction, where an m vitro method generates, inter alia, specific antibody.

In addition, such in an vitro method would allow for the generation of neutralizing antibodies against pathogenic antigens unacceptable for use in vivo because they are foreign animal diseases. Furthermore, such an in vitro system would be useful for screening vaccine candidates that would ultimately reduce the number of clinical trials needed to find effective vaccines for diseases.

### SUMMARY OF THE INVENTION

The disclosure relates to generally to vaccine and antibody development.

In embodiments, a method of activating B-cells in vitro is disclosed including contacting isolated follicular dendritic cells (FDC) from a subject immunized with an antigen and isolated peripheral blood mononuclear cells (PBMC) from a subject, where the isolated. PBMC comprise naïve B-cells; incubating the contacted cells; and detecting changes in the expression of a subset of cluster of differentiation (CD) antigens in the B-cell population: where detection of changes in the expressed subset of CD antigens correlates with activation of naïve B-cells.

In one aspect, B-cell activation comprises differentiation of said naïve B-cells to memory B-cells.

In another aspect, the antigen includes Rift Valley Fever Virus (RVFV) subunits, Epizootic Hemorrhagic Disease Virus subunits, Influenza Virus subunits, ovalbumin, dextran, Transmissible Spongiform Encephalopathy prion proteins, and Alzheimer's disease mutated amyloid precursor protein (APP) and presenilins 1 and 2, SARS virus subunits, HIV subunits, Bovine Spongiform Encephalopathy prion proteins, SARS virus subunits, Avian influenza Virus subunits, West Nile Virus subunits, Keyhole Limpet Hemocyanin, LPS, Bluetongue Virus subunits, Porcine Epidemic Diarrhea Virus (PEDV) subunits, and combinations thereof.

In a related aspect, the antigen is an RVFV subunit, comprising the amino acid sequence as set forth in SEQ ID NO: 1.

In another aspect, the subject is a mammal. In one aspect, the method further includes contacting antigen presenting cells (APC) and T-cells with naïve B-cells prior to contacting naïve cells with FDC.

In another aspect, the subset of CD antigens includes CD6, CD9, CD10, CD11b, CD11c, CD19, CO20, CD21, CD22, CD23, CD24 CD25, Cd26, CD27 CD28, CD30, CD32, CD35, CD37, CD38, CD39, CD40, CD45RO, CD45RA, CD45RB, CD49b, CD49c, CD49d, CD50, CD52, CD57, CD62L, CD69, CD70, CD72, CD73, CD74, CD75, CD76, CD79α,β, CD80, CD83, CDw84, CD85, CD86, CD89, CD97, CD98, CD119, CDw121b, CD122, CD124, CD125, CD126, CD127, CD130, CD132, CD135, CDw137, CD138, CD139, and combinations thereof.

In a related aspect, the change is a reduction in B-cells expressing CD19 antigen.

In one aspect, the change is an increase in B-cells expressing CD11b antigen, CD11c antigen or both CD11b antigen and CD11c antigen. In another aspect, the FDC supports proliferation of B-cells in vitro.

In another embodiment, a method of promoting antibody production in hybridoma cells is disclosed including fusing a melanoma cell with splenic B-cells of a subject immunized with an antigen; cloning the resulting fused cells; contacting isolated follicular dendritic cells (FDC) from a subject and the cloned fused cells and optionally removing said FDC from said cloned fused cells; and determining the amount of antibody produced between fused cells contacted with FDC and non-contacted fused cells, where ELISA OD values between about 0.07 to about 0.1 indicate antibody production against the antigen.

In a related aspect, subsequent removal of the FDC results in decline of the antibody production from the contacted fused cells, in a further related aspect, the contacted fused cells subsequent to FDC removal express activation-induced cytidine deaminase (AID) in another related aspect, contacted fused cells subsequent to FDC removal undergo somatic hypermutation, class switch recombination or a combination thereof.

In another aspect, the antigen is selected from the group consisting of Rift Valley Fever Virus (RVFV) subunits, Epizootic Hemorrhagic Disease Virus subunits, Influenza Virus subunits, ovalbumin, dextran, Transmissible Spongiform Encephalopathy prion proteins, and Alzheimer's disease mutated amyloid precursor protein (APP) and presenilins 1 and 2, SARS virus subunits, HIV subunits, Bovine Spongiform Encephalopathy prion proteins, SARS virus subunits, Avian Influenza Virus subunits, West Nile Virus subunits, Keyhole Limpet Hemocyanin, LPS, Bluetongue Virus subunits, Porcine Epidemic Diarrhea Virus (PEDV) subunits, and combinations thereof, in a related aspect, the antigen is an RVFV subunit, comprising the amino acid sequence as set forth in SEQ ID NO:1.

In one aspect, an isolated, activated B-cell produced by the method above is disclosed.

In another aspect, an isolated hybridoma cell produced by the method above subsequent to contacting said FDC is disclosed.

In one embodiment, a FDC cell line AY3 having ______ accession no. ______ is disclosed.

In another embodiment, an immunogenic agent is disclosed.

### BRIEF DESCRIPTION OF THE DRAWINGS

FIG. 1 shows serum titres in vaccinated sheep on day 28 against Peptide 1, Peptide 2, and Peptide 3.

FIG. 2 shows the generation of Antigen-Specific B Cells.

FIG. 3 shows phenotypic and functional characterization of sheep FDC Cultures. FDCs express lineage markers in vitro. Control staining is shown in dotted lines, whereas FDCs are shown to express CD35, CD21, PrP, and CD40, but not the B cell marker CD85.

FIG. 4 shows proliferation of sheep B cells co-cultured with FDC cell lines for 24 or 72 hours. FDCs clearly supported significant enhancement in B cell proliferation.

FIG. 5 shows cultured Human Follicular Dendritic Cells: (a,b) Human FDCs 24 hours post-isolation. Note the significant number of free lymphocytes sell adhering to the surface of the FDCs. (c,d) Human FDCs 7 days post isolation. At this stage, all free lymphocytes have been removed, and the adherent cells are beginning to display typical FDC morphology.

FIG. 6 shows the proliferation of Human B cells in the presence and absence of cultured human FDCs. B cells were purified from normal human blood, and incubated in the presence (dotted bars) or absence (hatched bars) of FDCs in addition to 0 ug/ml LPS (A), 0.01 ug/ml LPS (B), 0.1 ug/ml LPS (C), or 1.0 ug/ml LPS (D). Lines AM1 and AY3 demonstrated the greatest induction of proliferation.

FIG. 7 shows that InAcT stimulates cytokine release by wild type macrophages but not Mal/MyD88 −/− macrophages.

FIG. 8 shows that InAcT stimulates cytokine release by TLR4 expressing HEK293 cells.

FIG. 9 shows in-vitro release of the antigen from InAcT formulation.

FIG. 10 shows that removal of RapidX abrogates elevated production of antibodies

FIG. 11 is a graph showing average PBMC proliferation, via BrdU assay, after 72 hour co-culture with FDCs (n=7).

FIG. 12 is a graph showing average percent CD19 positive cells (n=6).

FIG. 13 is a graph showing average percent antigen positive cells (n=6).

FIG. 14 are photographs of culture cells. On the left, human FDC line AY3 co-cultured with hybridoma A7.2 (anti-AEV). In co-culture, hybridomas tend to associate with individual FDCs. On the right, hybridoma A7.2 is evenly suspended in the media.

FIG. 15 is a graph showing that addition of FDCs promotes elevated Ab production against specific Ags. Numbers on the vertical axis represent OD measurements obtained from the ELISA Antigen 1: rAEV protein VP1. Antigen 2: rAEV protein VP3. Antigen 3: AEV live virus vaccine.

FIG. 16 is a graph demonstrating that removal of FDCs abrogates elevated production of antibodies. Antigen 1: rAEV protein VP1. Antigen 2: rAEV protein VP3. Antigen 3: AEV live virus vaccine.

FIG. 17 is a flow chart illustrating the use of monocyte derived dendritic cells to increase B cell activation and CD4 T cell activation upon addition of antigen in vitro.

### DETAILED DESCRIPTION OF THE INVENTION

Before the present composition, methods, and methodologies are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “a nucleic acid” includes one or more nucleic, acids, and/or compositions of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs. Any methods and materials similar or equivalent to those described herein can be used in the practice or testing of the invention, as it will be understood that modifications and variations are encompassed within the spirit, and scope of the instant disclosure.

As used herein, “about,” “approximately,” “substantially” and “significantly” will be understood by a person of ordinary skill in the art and will vary in some extent depending on the context in which they are used. If there are uses of the term which are not clear to persons of ordinary skill in the art given the context in which it is used, “about” and “approximately” will mean plus or minus <10% of particular term and “substantially” and “significantly” will mean plus or minus >10% of the particular term.

As used herein, “naïve B cells” are B lymphocytes that have not been activated by antigen.

As used herein, “subject” means animal, including mammals, such as ruminants and primates, including cows, deer, horses, mice, chickens, pigs, monkeys, sheep, and humans.

Emerging diseases are widely considered the greatest threat to public health. Over the past half-century, the majority of emerging; diseases have arisen as a result of the zoonotic transmission from either wildlife or domestic animals to the susceptible human population. Recent examples of diseases which have spread from animals to humans include Bovine Spongiform Encephalopathy, SARS, Avian Influenza, West Nile Virus, and a variety of previously characterized diseases that have spread to the domestic animal population.

In order to understand the interaction of these diseases in reservoir populations, and devise appropriate control strategies, it is critical to understand the nature of the disease process in these natural host species. Unfortunately, immunological tools to characterize immunity these outbred species are difficult to develop, and the species themselves complex to work with in controlled environments.

The sheep is a domesticated small ruminant, well suited to research environments, and more is known regarding the function and development of the sheep immune system than any other species. Thus, the sheep represents an effective experimental model to understand the physiological response of the immune system to disease. What is needed is to develop novel tools, reagents, and data regarding the function and behavior of the ruminant immune system, including ways in which it differs from murine and human paradigms. One way to achieve this is to define systems to measure B cell responses.

Recent events have sensitized American society to the threat of biological agents as weapons. Antibodies form a first and highly-effective line of defense against these and other pathogens in affected individuals: current challenges exist in the appropriate means to develop and provide those antibodies as a strong therapeutic regimen. Although effective, active vaccination relies upon the ability of the host to survive challenge with the infectious agent, or alternatively identification of a harmless alternative capable of inducing long-lasting immunity. In contrast, passive vaccination with intravenous antibody is effective both in preventing infection and in promoting the resolution of existing infection with various pathogens. A current challenge is to develop the therapeutic means to produce and test large quantities of protective antibodies for use in humans.

As disclosed herein, an in vitro follicular dendritic cell culture as a functional model of lymph node germinal center activity may be used to induce the production of antigen-specific B cells and secreted antibody in cell culture from virtually any species. While production of specific antibody against well defined antigens commonly used in immunology is disclosed, the technology may be extended to virtually any antigen, eliminating the need for either human or murine donors for future antibody production, including antibody production in diverse species.

In embodiments, co-cultures of human B cells and FDCs are used to produce antigen-specific B cells in response to select antigens. In a related aspect, heterohybridomas of antigen-specific B cells stimulated in vivo and in vitro may be produced in order to define the ability of in vitro immunized B cells to generate high affinity antibodies capable of blocking infection in vitro.

As disclosed herein, FDC lines from humans may be tested for their ability to support: antibody production in vitro. These antibodies may be tested for efficacy in vitro. In one aspect, a fully-human system may be developed for production of human polyclonal antibodies against multiple antigens, including the generation of new antibodies for research and diagnostics. In a related aspect, production of neutralizing human monoclonal antibodies that may be directed against the various biological agents is disclosed. Such monoclonal antibodies may be used in basic research, diagnostics, and investigated for therapeutic efficacy as a passive vaccine strategy for this biodefense agent. The work is unique, in that it relies upon development of sensitized B cells de novo from normal human peripheral blood. Finally, as will be recognized by one of skill in the art, establishment of the utility of the FDC cultures will further lead to new tools to assess vaccine efficacy in vitro, permitting development of rapid screening methodologies for development of new vaccine targets against pathogens of humans and animals.

Diseases such as cancer, autoimmune disease, and neurological disorders have plagued humans for centuries. Recent work in the field of immunology has shown tremendous promise in all of these areas by incorporating the use of monoclonal antibodies into treatment regimes. Humanized monoclonal antibodies have recently been developed that reduce immunogenicity in treated individuals, and improve target recognition. However, major roadblocks remain and current treatments are only moderately effective. Novel technologies are needed to move the use of monoclonal antibodies into mainstream use in medicine.

There are currently approximately 30 therapeutic monoclonal antibodies available in the Europe and the United States. Monoclonal antibody-based therapy has shown tremendous promise in the treatment of various types of cancer including colorectal cancer, B cell non-Hodgkin's lymphoma, and pediatric leukemia among others; even in patients that do not respond to traditional cancer treatments. In addition, antibody therapy has also shown encouraging results in patients with autoimmune diseases such as rheumatoid arthritis. Perhaps the most exciting use of monoclonal antibodies is in the field of neurological disorders including treatment of Alzheimer's and multiple sclerosis. Improved delivery systems have been developed that permit access across the blood-brain barrier, and offer exciting new hope in the treatment of these diseases. A current challenge remains in the production of sensitized B cells for production of fully human monoclonal antibodies.

To overcome this difficulty, a number of options have been explored. Firstly, serum antibody and B cells may be collected from human donors who have been previously exposed to the infectious agent. Clearly, the supply of these immune sera is limited, and ethical concerns preclude the active production of specific antisera by immunization. More recently, transgenic animals have been produced which generate fully human antibodies in response to immunization. While this technology has been successfully employed, the production of these transgenic animals is laborious, complex, and to date has been difficult to achieve at commercial Furthermore, concerns remain about the potential for zoonotic infections to be transmitted with any therapeutic products harvested from these engineered animals. A third option is the development of humanized antibody by genetic engineering or generation of“heterohybridomas” using murine fusion partner cell lines with B cells derived from humans. Once again, these procedures are laborious and expensive to achieve, and still rely upon in vivo immunization to produce specific antibodies. This is clearly impossible with highly infectious agents and other bacterial toxins known as “superantigens”, which produce sufficiently vigorous immune responses to overwhelm the immune system and cause death. An in vitro alternative to the generation of specific antibody is therefore required.

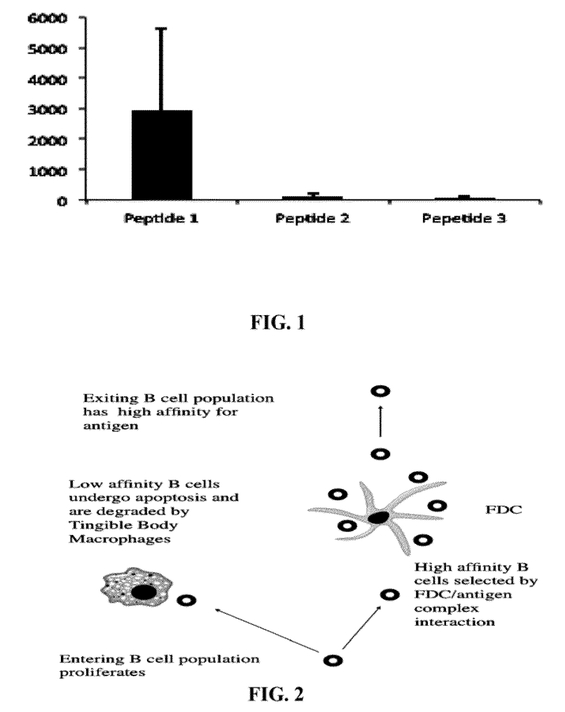

Recent data has used ex-vivo transformed human tonsillar B cells to generate fully human monoclonal antibodies potentially undergoing somatic hypermutation, but this method still relies upon a ready supply of B cells from secondary lymphoid tissue, rather than more accessible blood. During au immune response resulting in antibody production, the key cellular interactions take place within specialized regions of lymph nodes known as Germinal Centers (GCs) (FIG. 2).

While not being bound by theory, it is clear that B cells alone cannot be activated in culture to produce antibody. In fact, high-affinity antibody appears to be due to a specific interaction between a specific auxiliary cell type known as Follicular Dendritic Cells (FDCs). As compared to traditional dendritic cells, relatively little is known about the basic biology of FDCs.

As disclosed herein, phenotypically and functionally defined follicular dendritic cells from sheep, deer, cattle, mice, and humans have been successfully cultured, including that such FDC effectively support B cell proliferation and differentiation in vitro. These cultures are ideally suited to the identification of factors and responses to vaccine candidates, especially when combined with pre-existing technologies focused on defining T cell responses. As disclosed herein, the methods as described define appropriate protocols to support the de novo activation of human B cells in vitro for the ultimate production of fully human monoclonal antibodies.

While germinal centers (GC) contain both B and T lymphocytes, as well as macrophages, the central cell type which defines B cell follicles in lymph nodes and ectopic lymphoid accumulations is the FDC. In many respects, the FDC appears similar to fibroblasts, and while not being bound by theory, it is speculated that the FDC is derived from a fibroblast precursor. Notwithstanding, FDCs can be differentiated from fibroblasts through their relatively high expression of complement receptors CD21 and CD35. It is their expression of complement receptors, particularly CD21, which permits them to accumulate antigen complexes on their cell surface thereby contributing to B cell proliferation and differentiation during the antibody response. Many of these complexes appear to be secreted as membrane-bound microvesicles called iccosomes. Although the precise function of iccosomes remains unclear, it is believed that these specialized exosomes may in fact participate in amplification of the immune response. Regardless, it is clear that the FDC-B cell interaction is crucial for generation of a high-affinity antibody response.

In embodiments, methods have been developed to duplicate active GCs in cell culture, which can be exploited to generate high-affinity antibodies in vitro. This technology eliminates the need for living hosts in the generation of polyclonal and monoclonal antibodies and is particularly well suited for production of neutralizing antibodies against pathogenic substances unsuited for use in live animals.

An overall real of the disclosed process involved (a) Characterizing the development and phenotype of circulating B cell subsets in sheep, (b) Mapping the phenotype of memory B cells in sheep (c) Defining a simple, flow-cytometry based procedure to measure B cell memory in sheep and (d) Testing, the efficacy of an in vitro FDC culture system to support B cell proliferation and expansion in vitro.

In embodiments, B cell populations may be identified and differentiated by their CD antigens. For example, they may be defined as CD21^{lo}/CD1b^{neg}/CD11^{pos}; CD21^{neg}/CD11b^{pos}/CD11c^{pos}; and CD21^{pos}/CD11b^{neg}/CD11c^{neg}. As disclosed herein, CD21^{neg} B cells develop postnatally. Further, long term labeled CD21^{pos} B cells undergo cell division and downregulate CD21 expression in vivo. Moreover, CD21^{neg} cells express a surface phenotype consistent with memory B cells and are elevated in immunized subjects. Thus, while not being bound by theory, naïve and memory B cells may be defined based on expression of complement receptors.

Based on the observations as described herein: 1) Sheep naïve and memory B cells can he defined based on expression of complement receptors. Given the lack of expression of IgD in the sheep, this provides an important tool to monitor immune responses in domestic animals. 2) Directly labeled antigen can be used to monitor the development of antigen specific B cells in outbred animals in the absence of cell culture techniques. 3) Follicular Dendritic Cell (FDC) cultures support B cell, proliferation ex vivo, including the apparent differentiation from a naïve to memory phenotype.

In embodiments, the development of an in vitro method to generate antigen-specific B cells producing high affinity antibodies against target antigens is disclosed. Once isolated, these antibodies may be produced using a variety of methods (heterohybridomas, cloning and recombinant expression) for therapeutic and diagnostic use in humans.

There exists an immediate and pressing need for production of large volumes of human polyclonal antisera for biodefense as well as an ongoing need for other human conditions (i.e. antivenin). To date, the most effective means for producing these antibodies lies in isolating antigen-specific B cells from previously sensitized humans. For many infectious agents (SARS, HIV), it is not feasible to vaccinate humans for production of new antibodies, and alternatives are needed to generate antigen-specific B cells. An added benefit of the instant technology is that self-tolerance may be overcome, thus affording a means to produce ex vivo antibodies specific for tumor antigens, protein aggregation diseases such as the Transmissible Spongiform Encephalopathies and Alzheimer's disease, as well as other self-antigens.

To develop methods to examine the interaction of FDCs with B cells in domestic animals, the present disclosure describes a method to selectively culture sheep FDCs from normal and infected animals. Briefly, this method is a refinement of previous reports used to establish stable FDC lines from cattle and humans. This technology has been used successfully in murine systems were phenotypically and functionally defined FDCs can be cultured, capable of amplifying antibody production in vitro. In addition, cultured FDC lines produced in this way were demonstrated to promote somatic hypermutation in vitro, suggesting that they may be used to produce high-affinity antibody directed against specific antigens when immunized in vitro.

In one aspect, using follicular dendritic cell cultures for the de novo generation of antigen-specific B cells from naïve individuals, and protocols for the isolation of heterohybridomas specific for these target antigens may be carried out by the methods as described herein. In one aspect, the well-defined antigens (e.g., ovalbumin and dextran) may be used. In another aspect, production of neutralizing monoclonal antibodies against the NIH Priority Agent Rift Valley Fever Virus is disclosed. While Dextran may stimulate T-dependent responses, obviating the need for T-cell help, ovalbumin requires antigen processing and associated T cell help for production of highly-specific B cells, including somatic hypermutation to generate high-affinity antibodies. In one aspect, a baculovirus-produced Rift Valley Fever vaccine for domestic animals may be used to produce fully human neutralizing antibodies capable of inhibiting viral infection in vitro. Such recombinant antigens may be used to immunize B cells the in vitro culture system as disclosed, where the system may be screened for monoclonal antibodies capable of neutralizing Rift Valley Fever Virus infection in vitro.

While not being bound by theory, in vitro cultures of FDCs and normal peripheral blood leukocytes may be induced to produce high-affinity antibodies. Previous data has demonstrated the ability of lymph nodes “ex vivo” to produce antibodies in response to stimulation with protein antigen, and that murine FDC cultures support the production of antibody from in vivo stimulated B cells. In one aspect, a method to repeatably isolate FDCs from lymph nodes of sheep is disclosed.

As an initial step, the ability for in vitro FDC cultures to induce the de novo production of antigen-specific B cells is defined. Previously-developed assays may then be used to define the phenotype and frequency of antigen-specific B cells. In order to confirm that affinity maturation processes are active, PCR may be used to confirm that somatic hypermutation is occurring during B cell culture.

Briefly, antigen may be added to co-cultures of human peripheral blood cells and FDCs to induce antigen-specific B cell activation and differentiation. Donors may be screened by ELISA to confirm that no prior antibody is present, and flow cytometry and ELISPOT may be used to confirm the absence of antigen-specific memory B cells.

Previously, it was unclear as to whether B cells alone, or a mixture of B cells, T cells, and peripheral blood monocytes will produce optimal antibody responses, although data obtained from other species would suggest that T cells are absolutely required to induce affinity maturation and somatic hypermutation of antibodies in vitro. Although it would appear evident that T cell help will be required for optimal antibody responses, previous data has indicated that T cells are unnecessary for FDC-based B cell proliferation.

In one aspect, an in vitro system for de novo production of monoclonal antibodies is disclosed. To this end, it will be advantageous to immortalize B cell clones for screening and selection, as well as production of sufficient hybridoma supernatant. Once selected, immortalized B cell clones may be used to produce antibodies using either standard production protocols or established and well-defined recombinant technologies. In embodiments, established methods may be used to produce heterohybridomas from antigen-specific B cells stimulated in the in vitro system as disclosed herein.

Standard fusion methods may be used to produce human heterohybridomas which may then be screened by standard ELISA for the production of high-affinity antibody against target antigens. All monoclonals produced in this way may be tested for isotype in order to confirm that the system not only expands antigen-specific naïve B cells, but has the capacity to induce class-switching and hypermutation of immunoglobulins in vitro.

As described herein, these protocols are well characterized for the short-term production of antibodies in vitro, and have been used successfully for both human and sheep cells.

In embodiments, methods may include high-throughput screening and identification of vaccine targets in vitro that may be applied to both human and animal application. To that end, tools for the rapid identification of T cell epitopes using a Mass Spectrometry based approach ma be applied, including further technologies for identification of immunodominant B cell epitopes. In addition, an overall in vitro system is defined herein for rapid evaluation of vaccine candidates, including use of FDC cultures to evaluate B cell responses, including that such systems may be directly applied to in vitro screening of vaccine candidates directed against targets including Rift Valley Fever Virus, influenza, and vector-borne diseases of humans and animals.

In embodiments, B cell epitopes may mapped to generate subunit vaccines. In one aspect, surface proteins are targeted to increase likelihood of neutralizing antibody production. In a related aspect, neutralizing proteins may be identified from in vivo derived serum where available, as welt as from in-house “RapidX” assay. Sequences may be derived from GenBank and EMBL submissions (e.g., Rift Valley Fever, Epizootic Hemorrhagic Disease), direct sequencing (e.g., PEDV-SD Isolate), or a combination of both. Sequences may be analyzed for predicted B cell epitopes, and these epitopes compared across isolates.

In embodiments, RVFV Gn B cell epitopes are disclosed. In one aspect, B and T cell epitopes may be predicted using the Immune Epitope Database and Analysis Resource supported by the National Institute of Allergy and infectious Diseases, a component of the National Institutes of Health in the Department of Health and Human Services (Bethesda, Md.). For example, HLA-DRBI1 is genetically similar to ovine Class II DRB loci and used for the basis of predictive epitopes. Synthetic peptides may be commercially produced and used to analyze serological (antibody) specificity by ELISA, and to measure proliferation by Rift Valley Fever specific sheep T cells in vitro. In a related aspect, a peptide designated peptide-1:

(SEQ ID NO: 1)
SAHYLNNDGKMASVKCPPKYELTEDCNFCRQMTGASLKK

has been demonstrated to exhibit a specific B cell response (FIG. 1). The figure shows serum titers to RVFV peptides one month post vaccination. As shown, all titers are above background, however, peptide I generates the strongest immune response. In a related aspect, such epitopes also show a specific T cell response.

As disclosed herein, a method denoted as “RapidX” may be used to generate predictive responses. For example, as shown herein, cells from sheep were collected and immunized in vitro with RVFV peptide 1 using the RapidX Assay where proliferation of B and T cell subsets was assessed by flow cytometry at 4 and 7 days as an indicator of immunogenicity. See, e.g., Table 1.

TABLE 1

| | 0 Days | 4 Days | 7 Days |
|---|---|---|---|
| CD4+ T cells | − | ++ | ++++ |
| CD8+ T cells | − | +/− | + |
| B cells | = | ++ | ++++ |

In embodiments, the subunit approach is applied to PEDV. For example, a sequence of a South Dakota isolate was compared to as archived US isolate for Spike Protein (e.g. Genbank Accession NO. Q91AV1.1). Spike protein has been demonstrated to provoke strong immune responses against TGEV and PEDV following oral delivery. In one aspect, conserved immunostimulatory epitopes have been identified, and have been included in the DNA sequence to be inserted in a virus.

In embodiments, a baculovirus expression system may be used to generate subunits/selected epitopes. Such a system has the advantages of being well characterized and provides a relatively straightforward approach to production of recombinant protein or Virus-Like Particles. Unlike yeast systems, glycosylation is appropriate (one of skill in the art would recognize that such glycosylation may be dependent on the cell line chosen). Unlike mammalian culture systems, baculovirus systems may be cultured at normal room temperatures using well defined medium formulations.

In embodiments, adjuvants and select delivery systems may be combined with the subunit vaccine to improve efficacy of such vaccines. In general, adjuvants serve as sort-specific enhancers for delivery of antigen to the immune system, and serve to boost immune response through innate cell activation and antigen deposition. Such adjuvants should preserve the antigen, as well as provide strong co-stimulation to the immune system to boost antibody production. Adjuvants are critical to optimal immune response against recombinant subunit vaccines. The general purpose of delivery systems is to preserve the integrity of the vaccine following production and prior to administration, and target it directly to the appropriate organs and cells of the immune system. In embodiments, effective molecular adjuvants and oral delivery systems are disclosed herein.

With respect to delivery systems, nanotechnology based delivery are disclosed including zein-based delivery systems (e.g., nanoparticle, emulsions, micelles), where such delivery systems are inexpensive, plant-derived product, with proven safety profiles. Such systems may assist in transdermal delivery of encapsulated antigen or cytokines and man be lyophilized to stabilize vaccine formulations for extended periods. In one aspect, such a zein-based system is described in U.S. Pub. Nos. 2011/0091565 and 2012/0219600, each of which is incorporated by reference in its entirety.

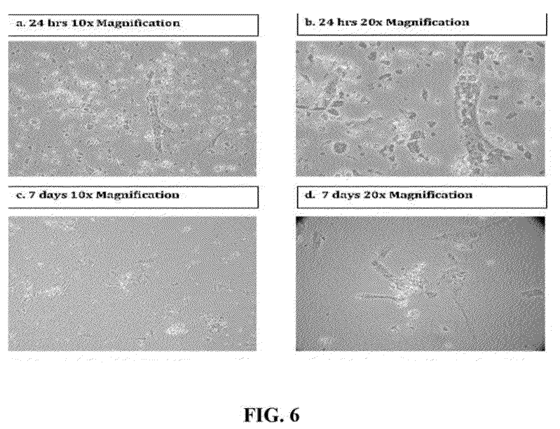

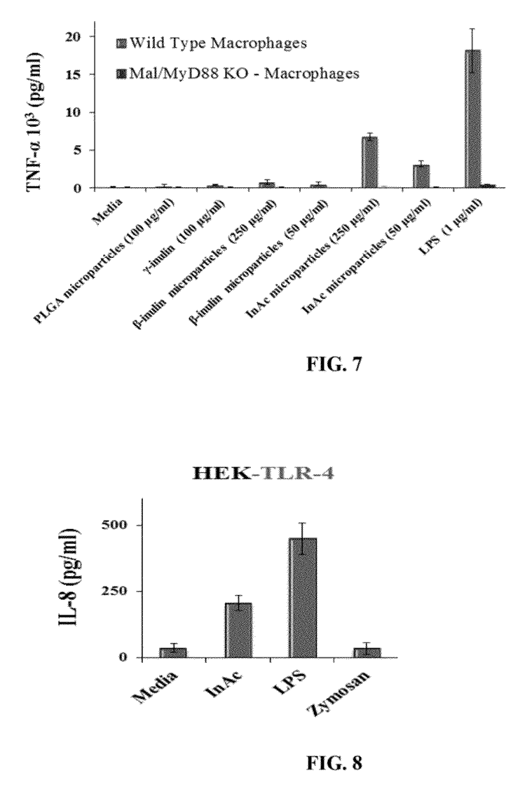

Another system may be an inulin-acetate (InAcT) based delivery system, which system comprises inexpensive raw material, encapsulates antigen for delivery to dendritic cells, stabilizes protein formulations following lyophilization, and is ideal for extended storage and shipping. In one aspect, the InAct also acts as an adjuvant, including that InAcT stimulates cytokine release by wild type macrophages but not by Ma1.MyD88 −/− macrophages (FIG. 7), stimulates cytokine release by TLR4 expressing HEK293 cells (FIG. 8) and enhances early response to RVFV vaccine with the IFA adjuvant ISA206. in one aspect, such an InAcT-based system is described in U.S. Pub. No. 2013/0195930, which is incorporated by reference in its entirety.

In one aspect, direct targeting of antigen to dendritic cells via InAcT adjuvant is disclosed. One advantage of this approach is dose sparing, which is demonstrated in Table 2.

TABLE 2
Mean Fluorescence Intensity

| S. No. | Treatment Groups | Counts | % of Green Cells |
|---|---|---|---|
| 1 | No Treatment | 4.06 ± 0.57 | 3.31 ± 1.61 |
| 2 | Ova in Solution | 13.18 ± 1.06 | 22.0 ± 2.68 |
| 3 | Ova Loaded SIMs | 324.16 ± 22.33 | 38.82 ± 0.71 |

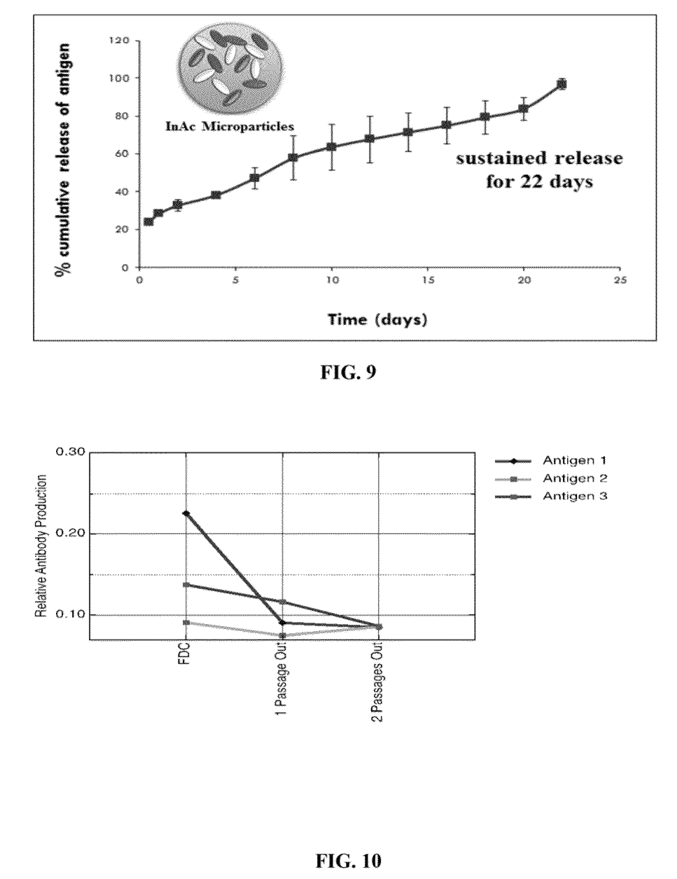

Other advantages include sustained release of encapsulated antigen, which results in sustained stimulation of an immune response. This has been demonstrated in FIG. 9, which shows the in vitro release of an antigen from InAcT formulation,

In embodiments, enhanced initial responses using the ISA206 adjuvant in combination with the InAcT antigen delivery system has been demonstrated. This promotes early development of neutralizing titres (⅔ animals demonstrated titres at 10 days post single injection).

As stated above, RapidX Assay has been used to generate predictive responses, including pre-development screening for protective epitopes. Further, the method may be used to generate monoclonal antibodies (or poly clonal sera) for development of DIVA companion ELISA assays for field use of vaccines.

In embodiments, RapidX promotes antigen-specific responses. For example, sheep PBMCs were purified and incubated in RapidX with a specific antigen (KLH) and frequency of antigen specific memory B cells and antigen-specific naïve B cells was assessed using flow cytometry.

RapidX promoted antigen-specific responses as seen in Table 3.

TABLE 3
RapidX and antigen specific responses.

| | Specific Antigen | Unrelated Antigen |
|---|---|---|
| Ag-Specific Memory Cells | ↑ | ↑ |
| Ag-Specific Naive Ceils | ↑↑↑↑ | ↓↓↓↓ |

In addition, the instant disclosure demonstrates that RapidX promotes increased viability and antibody production by murine hybridomas, potentially serving as a novel “helper” cell population in monoclonal antibody production. In one aspect, de-novo production of hybridomas from multiple species entirely in vitro may be accomplished using said system. In addition, RapidX promotes elevated antibody production and growth rate of hybridomas, including that removal of RapidX abrogates elevated production of antibodies.

The following examples are intended to illustrate but not limit the invention.

### EXAMPLES

### Example 1

### FDC Isolation

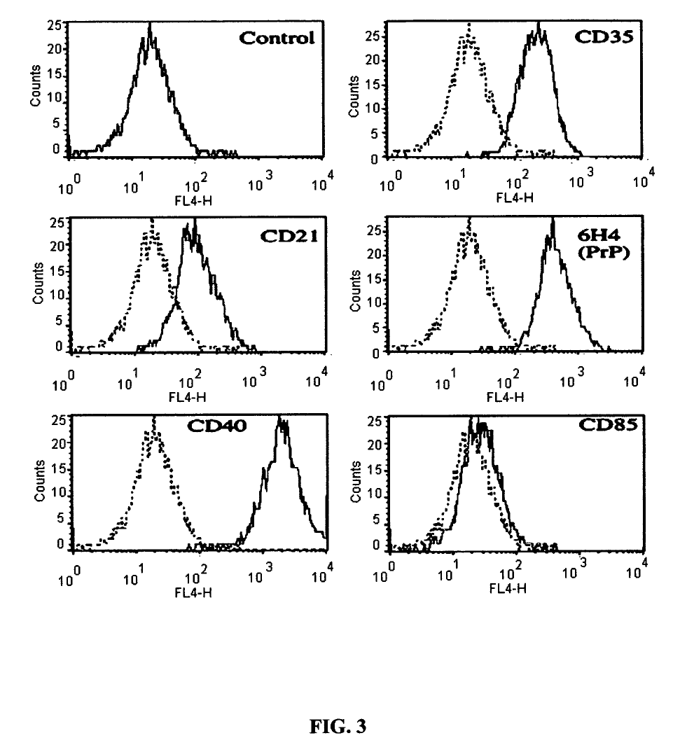

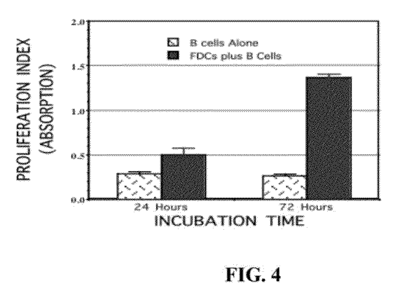

Briefly, a panel of FDC-specific monoclonal antibodies was used in conjunction with magnetic separation to purify FDCs from lymph node or tonsil suspensions. Cells were then separated using an AutoMACS magnetic sort apparatus, and plated for culture in rich tissue culture media containing 10% Fetal Calf Serum (FIG. 3). These cells morphologically resemble FDCs in culture and express the cell surface markers CD21, CD40, and CD35 which are distinct for FDCs, but not fibroblasts (FIG. 4). More importantly, these cell lines induce the selective proliferation of naïve B cells in culture, and their differentiation into phenotypically-defined memory B cells. A next necessary step was to define the utility of this method to support human FDC culture.

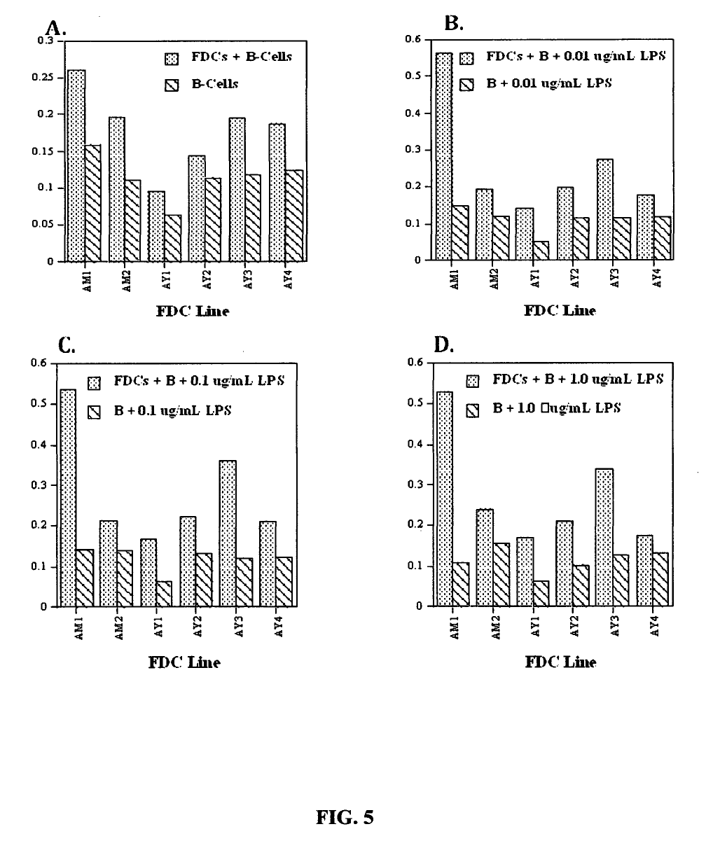

To accomplish the isolation and characterization of human FDCs, tonsils were used as a source of GCs and FDCs isolated using magnetic separation as previously used for sheep cultures. Human FDCs were cultured in Iscove's Modified Dulbecco's Media containing 10% Fetal Calf Serum and 0.1 μg/ml of E. coli LPS, and characterized based on morphology and phenotype as before Human FDCs exhibited similar morphological characteristics to sheep FDCs in culture, growing as adherent lines and specifically supporting B cell proliferation in vitro (FIGS. 5, 6). Addition of suboptimal levels of LPS further enhanced human B cell proliferation by two of the isolated lines.

Basically, the method mimics the stimulation of B and T cells that occurs during an immune response in germinal centers. The method provides for culturing of FDCs, and maintains their capacity to stimulate and differentiate B cells.

Culture and differentiation of monocyte derived dendritic cells (MDDCs) is well characterized, where isolation of FDCs is far less characterized, and only sparsely reported. The RapidX method as disclosed reproducibly isolates primary FDCs, which FDCs may be cultured for months and continue to support B cell function. For example, in co-culture, B cells tend to associate with individual FDCs. Further, the method generates memory B Cells in vitro. The effects of FDC and B-cell interactions may be seen in Table 4.
Table 4, Fold-Increase and Decrease in Expression of CD21, CD11b, and CD11c Induced by

TABLE 4
Fold-Increase and Decrease in Expression of CD21, CD11b,
and CD11c Induced by FDC-B Cell Co-Culture.

| Time in Culture | % CD21 | % CD11b | % CD11c |
|---|---|---|---|
| 0 Hours | 63% | 6% | 37% |
| 72 Hours | 27% | 13% | 45% |
| Up/Down Regulation | [symbol] 0.4 | [symbol] 2.1 | [symbol] 1.2 |
| 0 Hours | 70% | 13% | 49% |
| 72 Hours | 19% | 28% | 53% |
| Up/Down Regulation | [symbol] 0.3 | [symbol] 2.0 | [symbol] 1.1 |

### Example 2

### The Ability of FDCs to Promote B Cell Proliferation and Antigen Specific Responses In Vitro

### Ovine FDC Isolation

Ovine FDCs were previously isolated from lymph nodes and were available for use. Briefly, after removing connective and adipose tissue surrounding the lymph node and removing the capsule, the lymph node was cut in slices and digested for 30 minutes at 37° C. with intermittent shaking using 0.05% collagenase and DNase, in Iscove's modified Dulbecco's Medium (IMDM) containing 0.04% BSA, and 2% EDTA. The disassociated cells were collected, via centrifugation at 300×g for 7 minutes, in a sterile 50 ml conical centrifuge tube (Fisher) and the remaining tissue was subjected to two additional cycles of enzymatic digestion for 20 minutes at 37° C. with 0.05% collagenase, 0.04% DNase in IMDM supplemented as described above. The centrifuged cells were passed through 70-μm cell strainers to filter unwanted, larger debris and to aid in yielding a single-cell suspension. Cell suspensions were kept on ice for the duration of the procedure. After passage through the filter, FDCs were collected by centrifugation at 300×g for 7 minutes. Single cell suspensions were washed three times at 300×g for 7 minutes in PBS containing 2 mM EDTA and 1% fetal bovine serum (FBS) and counted on a Coulter Particle Counter (Beckman Coulter—Z1). Cells were resuspended in complete IMDM culture media supplemented with 10% EBS prior to positive selection.

### Human FDC Isolation

Human FDCs were previously isolated from tonsils and were available for use.

### Positive Selection of FDCs by AutoMACS

Collected digested nodes were incubated with either antibody 2-137 or 6-184- (FDC specific hybridomas) at a concentration of 10⁸ cells per ml of the antibody supernatant. The cells were kept on ice for 45 minutes, followed by three successive washes in PBS+1% FBS. Cells were then incubated for 15 minutes at 4° C. with isotype specific microbeads (Rat anti-mouse RAM), (Miltenyi Biotec, Gladbach, Germany) at a concentration of 20 μl/10⁷ cells based on the manufacture's recommendations. Cells were washed an additional three times. Cells were resuspended in 5 ml PBS+1% FBS and sorted using an autoMACS magnetic cell sorter (Miltenyi Biotec) using the Possel mode program. The positively selected eluted cells were washed immediately in culture media, and resuspended in complete IMDM culture media supplemented with 10% PBS.

### FDC Cell Culture

Single cell suspensions were prepared as previously described, in a solution of modified Iscove's Modified Dulbecco's Medium (IMDM), supplemented with 10% FBS, 10 μl/ml non-essential amino acids, 100 IU/ml penicillin, and 100 μg/ml streptomycin. Cells were plated in either 6-well plates or 96-well plates and incubated at 37° C. and 5% CO₂.

### Maintenance of Cultured Cells

Cultures were examined daily by inverted phase contrast microscopy, with morphology, medium color, and cell density noted. Cells were fed with new media every 3 to 4 days and were harvested and split when they were at least 75% confluent.

### Passage of FDCs

FDCs were passaged once they were at least 75% confluent. Harvesting of cells was performed enzymatically by using a 0.25% trypsin solution containing 0.2 g/L EDTA (HyClone). The medium was removed from the culture plate, and the cells were washed with DPBS warmed to 37° C. The cells were then incubated at 37° C. for 5-10 minutes with enough 0.25% trypsin-EDTA solution to cover the bottom of the culture plate (0.5 ml for 6-well plates and 50 μl for 96-well plates). When the cells were in suspension, IMDM-10% NXBS was added. An appropriate volume of cells was transferred to a new plate and incubated at 37° C. with 5% CO₂. FDCs used in the experiments were passaged anywhere from 5-20 times. The number of passages did not affect the performance of the FDCs.

### Experimental Animals

To test the efficacy of an in vitro FDC culture system to support B cell proliferation and expansion, three healthy adult outbred sheep were obtained from the South Dakota State University Sheep Unit. Sheep were immunized on day 0 and day 14 with the T-dependent antigen keyhole limpet hemocyanin (KLH) and the T-independent antigen dextran, using 100 μl of antigen in combination with Freud's Adjuvant for a final volume of 2 ml. The appearance of antigen-specific B cells was monitored by dual-color staining and analyzed via flow cytometry. FDCs were isolated from lymph nodes by positive selection, and their ability to support B cell proliferation defined in vitro.

Isolation of Peripheral Blood Mononuclear Cells (PBMC) from Whole Blood

White blood cells (WBCs) were isolated from whole blood by PERCOLL™ density gradient centrifugation. Twenty ml of peripheral blood was collected in a syringe containing 2 ml of 0.5 M EDTA as an anticoagulant. The blood was then diluted 1:1 with PBS supplemented with 2 mM EDTA in a 50 ml centrifuge tube and centrifuged at 1750×g for 15 minutes with the brake on. The buffy coat containing the white blood cells was collected from the top of the red blood cells, transferred to a 50 ml centrifuge tube, and diluted with PBS containing, 2 mM EDTA for a total volume of 40 ml. Ten (10) ml of the buffy coat was laid carefully over 3 ml of 60% PERCOLL™ diluted in Hanks Balanced Salt Solution in a 15 ml centrifuge tube and centrifuged at 1750×g with no brake. Using a Pasteur pipette, the white-cell layer was collected from the top of the PERCOLL™, transferred to a 15 ml centrifuge tube and diluted with PBS-EDTA for a total volume of 10 ml. The cells were washed twice with PBS-EDTA and centrifuged 450×g for 7 minutes prior to cell counting,

### Ovine PBMC and FDC Co-Culture

Ovine FDCs were grown to at least 60% continency before the addition of PBMCs. PBMCs were isolated via density centrifugation using 60% PERCOLL™, resuspended in an appropriate volume of IMDM-10% FBS-0.1 μg/ml lipopolysaccharide (LPS) (Escherichia coli, serotype 0111:B4, Calbiochem, Cat. No, 437627) and evenly split between the wells for the experiment, striving for one million PBMCs per well. Cells were incubated at 37° C. and 5% CO₂ for 72 hours and then either a proliferation assay was performed or flow cytometry analysis with anti-sheep IgM-FITC (Southern Biotech).

### Ovine Cell Dual-Color Staining

For dual-color staining, cells were collected from the FDC co-culture plates, washed twice with PBS+1% FBS and plated in 96 well plates. To appropriate wells, 100 μl of either anti-sheep CD21, anti-sheep CD11b or anti-sheep CD11c was added and incubated for 10 minutes at 4° C. Cells were centrifuged and after two washes in PBS+1% FBS, 50 μl of FITC mouse anti-sheep IgM (diluted 1:1000 in PBS-FBS) was added to each well and incubated for 10 minutes at 4° C. Cells were centrifuged and after two washes in PBS+1% FBS, the cells were fixed in 100 μl of 1% paraformaldehyde (Sigma) in PBS and analyzed by flow cytometry. All centrifugation was performed using a Beckman GS-6KR Centrifuge set at 1200 RPM for 1 to 2 minutes. Fixed cells were stored at 4° C., covered, for up to one week and then transferred to tubes and analyzed using a dual-laser FACS Calibur flow cytometer and Cell Quest Pro software.

### Human PBMC and FDC Co-Culture

Ovine and human FDCs were grown to at least 60% confluency before the addition of PBMCs, PBMCs were isolated via density centrifugation using 60% PERCOLL™, resuspended in an appropriate volume of IMDM-10% FBS-0.1 μg/ml lipopolysaccharide (LPS) (Escherichia coli, serotype 0111:B4, Calbiochem, Cat. No. 437627) and evenly split between the wells for the experiment. Cells were incubated at 37° C. and 5% CO₂ for 72 hours and then either a proliferation assay was performed or flow cytometry analysis with APC mouse anti-human CD19 and FITC-labeled H1 influenza antigen (CDC-IRR).

### FITC Conjugation of Recombinant Utah H1pdm Influenza Antigen

A Pierce FITC antibody labeling kit was used for FITC conjugation. Briefly, 10 μl of borate buffer was added to 200 μl Utah H1pdm influenza antigen (1.0 mg/ml, CDC-IRR). All of the prepared protein was added to the vial of FITC reagent and pipetted up and down ten times until the dye was dissolved. The vial was briefly centrifuged and incubated at room temperature for one hour, in the dark. Two hundred (200) μl of purification resin was added to a spin column and centrifuged for 30-45 seconds at about 1000×g to remove the storage solution. All of the labeled reaction was added to the spin column and mixed with the resin by briefly vortexing. The column was centrifuged for 30-45 seconds at 1000×g to remove the free FITC and collect the purified protein that ran through the column. The labeled influenza was stored, protected from light, at 4° C.

### Human Cell Dual-Color Staining

For dual-color staining, cells were collected from the FDC co-culture plates, washed twice with PBS+ 1% FBS and plated in 96 well plates. To appropriate wells, FITC labeled recombinant H1pdm Utah influenza antigen and APC mouse anti-human CD19 antibody was added and incubated for 10 minutes at 4° C. Cells were centrifuged and after two washes in PBS+ 1% FBS, the cells were fixed in 100 μl of 1% paraformaldehyde (Sigma) in PBS and analyzed by flow cytometry. All centrifugation was performed using a Beckman GS-6KR Centrifuge set at 1200 RPM for 1 to 2 minutes. Fixed cells were stored at 4° C., covered, for up to one week and then transferred to tubes and analyzed using a dual-laser FACS Calibur flow cytometer and Cell Quest Pro software.

### Proliferation Assay

B cells and FDCs were isolated as described above and resuspended in complete IMDM culture media supplemented with 10% FBS and 0.1 μg/ml UPS (used as a mitogen). Duplicate 200 μl aliquots of the cell suspension were plated in a 96-well, flat-bottom plates. To each well was added one of three treatments: PBMCs alone, FDCs alone, or both PBMCs and FDCs. The cells were then incubated at 37° C. in the presence of 5% CO₂ for 72 hours.

After the three-day incubation, a BrdU colormetric immunoassay (Proliferation ELISA, BrdU, Roche Applied Science) was used to determine proliferation of the PBMCs by the FDCs in the presence of mitogens. This was measured by the incorporation of 5-bromodeoxyuridine (BrdU) during DNA synthesis. The manufacturer's instructions were adapted as follows:

After warming in a 37° C. water bath, 20 μl of the BrdU Labeling (working) Solution was added to the cells in each well of a 96-well plate. The plate was reincubated at 37° C. in a 5% CO₂ incubator for 2 hours. After incubation, the plate was centrifuged for 10 minutes at 1200 RPM. The supernatant was removed, the cells were dried using a hair dryer (medium setting), for 15 minutes. FixDenat (Kit-Bottle #2) was added (200 μl per well), and the plate was incubated for 30 minutes at room temperature in the dark. The FixDenat was removed thoroughly by a quick inversion of the plate, followed by tapping.

A freshly-prepared anti-BrdU-POD working solution was added at 100 μl per well before the plate was incubated for 1 hour at room temperature in the dark. The anti-BrdU solution was removed by a quick inversion of the plate. Wells were rinsed three times with 200 μl of washing solution. The Substrate Solution (Kit-Bottle #6) was added (100 μl per well), and the plate was then incubated for 20 minutes at room temperature in the dark. Sulfuric acid (H₂SO₄) was used as the stop solution; 25 μl was added to each well within 5 minutes prior to reading the plate to avoid overexposure. Results were acquired using an ELISA reader set at a wavelength of 450 nm.

### Antibodies and Antigens Used

For ovine cell dual-color staining, the following in-house primary antibodies were used at 100 μl per well: anti-sheep CD21 (mAb 2-87), anti-sheep CD11b (mAb 12-5-4), and anti-sheep CD11c (mAb 17-196). The secondary antibody, FITC mouse anti-sheep IgM (Southern Biotech), was added to each well at 50 μl per well (after 1:1000 dilution with PBS-FBS).

For human cell co-culture, influenza A (H1N1) pdm09 Control Antigen (A/California/07/2009 NYMC X-179A) BPL Inactivated (FR-187; CDC-IRR), whole virus, was added to appropriate wells at 100 μl per well.

For human cell dual-color staining, APC Mouse Anti-Human CD19 (BD Pharmingen) was added to appropriate wells at 20-25 μl per well, and FITC-labeled recombinant Utah H1pdm Influenza antigen (CDC-IRR) was added to appropriate wells (after 1:100 dilution with PBS-FBS) at 50 μl per well.

### Phosphate-Buffered Saline (PBS)

PBS was prepared by diluting a commercial 10× PBS stock (Fisher BP399-4). 100 ml of 10× PBS was added to 900 ml of distilled water to reach a final concentration of 0.04 M solution of phosphate buffered in 0.15 M saline, with pH adjusted to 7.40.

### EDTA-PBS

Two (2) triM EDTA-PBS was prepared by adding 4 ml of 0.5M EDTA to 996 ml PBS.

### Hanks' Balanced Salt Solution (HBSS)

One volume of a ten-fold concentrate HBSS (GIBCO Laboratories, Grand Island, N.Y.) was combined with 9 volumes of distilled water.

### 100% PERCOLL™ (Sterile)

Ninety (90) ml sterile PERCOLL™ (Amersham Biosciences) 1.131 g/ml, was diluted with 10 ml sterile 10× HBSS.

### Preparation of 60% PERCOLL™ (Sterile)

Sixty (60) ml of 100% PERCOLL™ was diluted with 40 ml 1× HBSS.

### Preparation of Complete Culture Medium

A 500 ml bottle of IMDM (BioWhittaker, IMDM) was supplemented with 5 ml of non-essential amino acids, 1 ml of 2-mercaptoethanol (final concentration: 0.1 mM), 5 ml penicillin/streptomycin solution (Fisher MT30002Li, final concentration: 100 U/ml penicillin and 100 μg/ml streptomycin). All media were stored at 4° C. and warmed to 37° C. before use.

### Paraformaldehyde

A 1% (w/vol) of fixative paraformaldehyde solution was prepared adding one gram of para formaldehyde (Sigma) to 50 ml distilled water, preheated to 65° C., with continuous stirring. The mixture was allowed to cool to room temperature, before a two-fold concentrate of PBS was added. NaOH solution was added drop-wise to completely dissolve the paraformaldehyde.

### BrdU Labeling Solution

A colorimetric commercial kit, the Cell Proliferation ELISA, BrdU (Roche Applied Science, Mannheim, Germany, cat #11647229001), was used. One (1) ml BrdU, (labeling Reagent Kit-Bottle #1), pre-warmed to 37° C. was added to 99 ml IMDM-10% FCS.

### Anti BrdU Labeling Solution

The anti-BrdU-POD working solution was prepared by adding 1 ml of the anti-BrdU-POD Stock Solution (Kit-Bottle to 99 ml of the antibody Dilution Solution (Kit-Bottle #4).

### Washing Solution

The washing solution was made by diluting the Washing Buffer Solution (Kit-Bottle #5) in sterile water at a concentration of 1:10.

### Development of Activated/Memory B Cells In Vitro Using an Ovine Model

To test the ability of FDC cultures to support B cell proliferation and expansion in vitro, an ovine model was used. Three healthy adult sheep were immunized with KLH and Dextran, and the appearance of antigen-specific B cells were monitored by direct binding of fluoresceinated antigen and analyzed with flow cytometry. In sheep, CD21 is expressed on mature, naïve B cells and, as the B cells become activated and differentiate into memory B cells, they become CD11b/CD11c positive. There was an average 0.47 fold decrease in the percent of CD21 expression in the cells (Table 5), indicating that there was a decrease in the percentage of naïve B cells. Also, there was an average 4.23 told increase in the percent of CD11b expression and an average 1.40 fold increase in the percent of CD11c expression (Table 5), suggesting that the B cells had become activated and potentially differentiated into memory B cells. It appears that FDCs trigger B cells to differentiate from a naïve to a memory phenotype ex vivo (Table 5).

TABLE 5
Fold increase and decrease in expression of CD21, CD11b and
CD11c induced by FDC-B cell co-culture (n = 3).

| | | | |
|---|---|---|---|
| Time in Culture | % CD21 | % CD11b | % CD11c |

| | | | |
|---|---|---|---|
| 0 Hours | 63% | 6% | 37% |
| 72 Hours | 27% | 13% | 45% |
| Up/Downregulation | −0.4 | 2.1 | 1.2 |
| 0 Hours | 73% | 5% | 37% |
| 72 Hours | 53% | 43% | 70% |
| Up/Downregulation | −0.7 | 8.6 | 1.9 |
| 0 Hours | 70% | 13% | 49% |
| 72 Hours | 19% | 26% | 53% |
| Up/Downregulation | −0.3 | 2.0 | 1.1 |

### FDCs Support Ovine B Cell Proliferation In Vitro

At 24 hours of co-culture, the B cells in the presence of FDCs have a proliferation index almost twice as high as that of the B cells alone (FIG. 4). At 72 hours, the proliferation index of those B cells with FDCs was approximately five times higher than the B cells alone (FIG. 4).

### Effects of FDCs on Human PBMC Proliferation

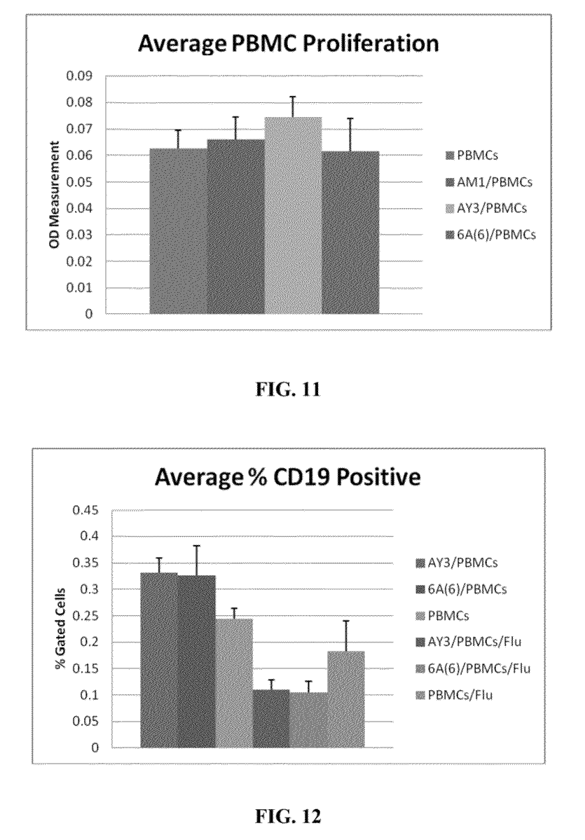

To test the ability of FDCs to support B cell proliferation, PBMCs were isolated from human blood and co-cultured with human or ovine FDCs and incubated for 72 hours before a BrdU assay was performed. A trend toward higher proliferation when PBMCs were in the presence of FDCs was revealed by the BrdU assay (FIG. 11). Individual variation among subjects was seen; however, the overall tendency was that PBMCs co-cultured with the human FDC line AY3 had the most increase in proliferation compared to all other conditions (FIG. 11).

### Effects of FDCs on Antigen-Specific Responses of Human PBMCs

To test the ability of FDCs to enhance antigen-specific B cell responses, PBMCs were isolated from human blood and co-cultured with human or ovine FDCs, with some cultures containing BPL inactivated influenza A (H1N1) pdm09 whole virus, and incubated for 72 hours before dual-color staining and flow cytometry analysis. CD19 is expressed on all B cells. There was a trend for a higher percentage of CD19 positive cells when PBMCs were co-cultured with FDCs, with an average of between 30-35% cells expressing CD19 in the presence of FDCs compared to less than 25% expressing CD19 in PBMCs alone (FIG. 12), suggesting that the total number of B cells increased in the presence of FDCs. However, the co-cultures containing whole influenza virus appeared to have a decreased percentage, ranging from about 10-30% reduction, of CD19 positive cells, (FIG. 12), implying that there was a decrease in the total amount of B cells.

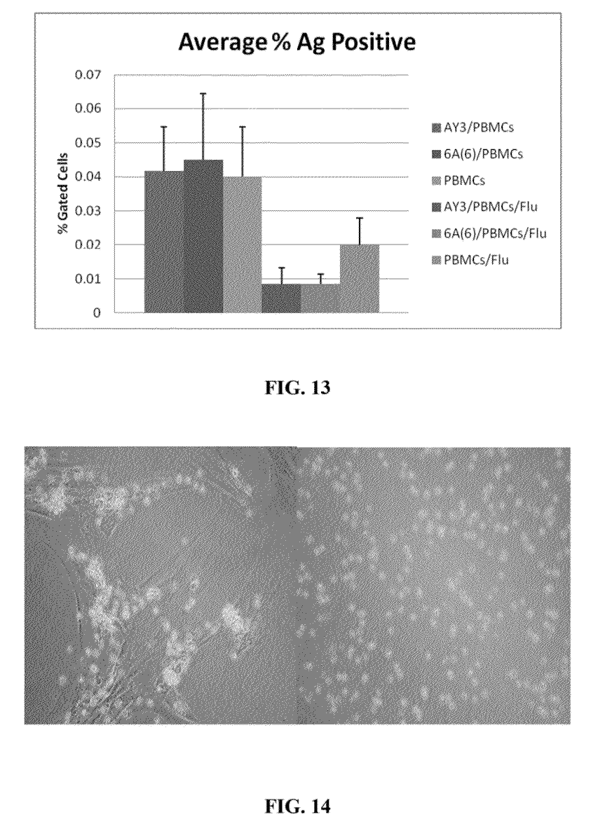

When comparing the percentage of influenza antigen positive cells, there were no significant difference between PBMCs alone and PBMCs with FDCs; in all cases about 4% of the cells were positive for influenza antigen (FIG. 13). Similar to the percent of CD19 positive cells, the co-cultures containing whole influenza, virus appeared to have a decreased percentage of antigen positive cells, ranging from 1-2% of the cells being antigen positive (FIG. 13). Results also demonstrated that all cultures that contained whole influenza virus had decreased percentage of both CD 19 and antigen positive cells (FIG. 12 and FIG. 13).

The FDCs displayed functional characteristics consistent with those in vivo, especially the ability to support B cell proliferation in vitro. The results from these experiments support the previous data that FDCs are able to support B cell proliferation in vitro, most notable in the ovine model.

To explore the potential for this system as a human model, human FDCs and PBMCs were used. Overall, there seemed to be a trend of higher proliferation of the PBMCs when FDCs were present, regardless of species.

### Example 3

### The Ability of FDCs to Promote Ig Production of Hybridomas

### Hybridoma Cell Line

The Sp2/0 cell line, developed by Schulman et al., was chosen as the fusion partner for immune spleen cells. These cells were maintained in IMDM-10% FBS until the day of fusion. A mouse was immunized against avian encephalomyelitis virus (AEV) using recombinant antigens VP1 (Bioclone) and VP3 (Bioclone) and then B cells were derived from the spleen. B cells were then fused with Sp2/0 cells using polyethylene glycol, generating hybridoma cells. The cells of some hybridoma clone positive wells with high titers of anti-VP1/VP3 were expanded and sub-cloned using the limiting dilution method. Cloning of hybridoma cells was performed in 96-well flat-bottom plates (Thermo) using complete RPMI medium with 10% FBS and incubated at 37° C. and 5% CO₂. Once sufficient sub-cloning was performed, cells were cultured in IMDM-10% FBS, and the level of FBS was slowly decreased to completely serum free media. The hybridoma cells were monitored regularly for titers of anti-VP1/VP3, using an ELISA. The A7.2 cell line was chosen to co-culture with FDCs because it was slowly losing titer as the percent of FBS was lowered.

FDC cell line

Human FDC line AY3 was isolated and cultured as previously described.

### Antigens Used

Recombinant AEV protein VP1 (1 μg/μl, Bioclone), recombinant AEV protein VP3 (0.5 μg/μl Bioclone), or AEV live virus vaccine (tremor Blend, Merial) were used to monitor antibody production by ELISA. VP1 and VP3 were diluted 1:1000 in Carbonate/Bicarbonate and used at 50 μl per well. The AEV vaccine was diluted 1:100 in Carbonate/Bicarbonate and used at 50 μl per well.

### ELISA

Antibody production was monitored by ELISA. Briefly, 96-well flat-bottomed plates (Thermo) were coated with either 50 μl of recombinant AEV Protein VP1 (1 μg/μl, Bioclone), recombinant AEV Protein VP3 (0.5 μg/μl, Bioclone), or AEV vaccine (Merial). Plates were incubated overnight, covered, at 4° C. Antigens were dumped off the plate, 100 μl blocking solution (5% dry milk) was added to each well, and incubated for one hour at room temperature. Wells were washed three times with 200 μl per well of PBST. Duplicate samples were added to each well at 100 μl per well and incubated for two hours at room temperature. Plates were washed three times with 200 μl per well of PBST. One hundred (100) μl of goat anti-mouse Ig (H+L)-HRP (Southern Biotech), diluted 1:1000 in 1% dry milk, was added to each well and incubated one hour at room temperature, in the dark. Plates were washed three times with 200 μl per well of PBST. 100 μl TMB substrate (1:1 dilution of kit components, Pierce) was added to each well and incubated for fifteen minutes at room temperature, in the dark. The reaction was stopped with 100 μl per well of 1N H₂SO₄ and plates were read at 450 nm with an ELISA plate reader.

### Antibody Isotyping

A Pierce Rapid ELISA mouse mAb Isotyping Kit (37503) was used to perform antibody isotyping, per kit instructions. Briefly, plate strips and TMB substrate were warmed to room temperature prior to use. Hybridoma supernatant was diluted 1:10 with Tris Buffered Saline (TBS). Samples were added to each well of the 8-well strip at 50 μl per well. Fifty (50) μl of goat anti-mouse antibody-HRP, included in kit, was added to each well and mixed gently. Plates were covered and incubated for one hour at room temperature. The contents of the plates were flicked off and the plates were washed vigorously three times with 250 μl per well of 1× Wash Buffer. TMB substrate was added to each well at 75 μl per well and incubated about 15 minutes at room temperature, in the dark. The reaction was stopped using 75 μl per well of stop solution and plates were read at 450 nm with an ELISA plate reader. Results were interpreted using the location map.

### RT-PCR

Total RNA was isolated from cells using an RNeasy Plus Mini Kit (Qiagen) and isolation of mRNA was performed using an Oligotex mRNA Spin-Column kit (Qiagen). The isolated mRNA was then used to generate cDNA using the Super Script First strand Synthesis System (invitrogen) at 42° C. for 50 minutes (TaKaRa Ex Taq DNA Polymerase). Primers used for analysis are listed in Table 6.

TABLE 6
Primer sequences used in RT-PCR analysis.

| Gene | Primer | Sequence |
|---|---|---|
| AID | Forward | 5′-CGTGGTGAAGAGGAGAGATAGTG-3′ |
| | | (SEQ ID NO: 2) |
| | Reverse | 5′-CAGTCTGAGATGTAGCGTAGGAA-3′ |
| | | (SEQ ID NO: 3) |
| GAPDK | Forward | 5′-TGTGTCCGTCGTGGATCTGA-3′ |
| | | (SEQ ID NO: 4) |
| | Reverse | 5′-CCTGCTTCACCACCTTCTTGAT-3′ |
| | | (SEQ ID NO: 5) |

GAPDH (IDT) was used to normalize mRNA expression, running 40 cycles of 98° C. for 10 seconds, 59° C. for one minute and 72° C. for one minute. AID (IDT) was amplified using 40 cycles of 98° C. for 10 seconds, 60° C. for 30 seconds and 72° C. for one minute.

### FDCs Support Hybridoma Growth In Vitro

To test the ability of FDCs to boost/support hybridoma growth in vitro, hybridoma cells were co-cultured with a human FDC line over a 5-month period of time. Hybridomas associated with the FDCs very intimately while hybridomas in culture alone were evenly suspended in the media (FIG. 14). This is consistent with observations in vivo with B cells and FDCs in the GC, as the close physical contact between B cells and FDCs is required for B cells to eventually differentiate into either plasma cells or memory B cells.

### FDCs Promote Ig Production by Hybridomas

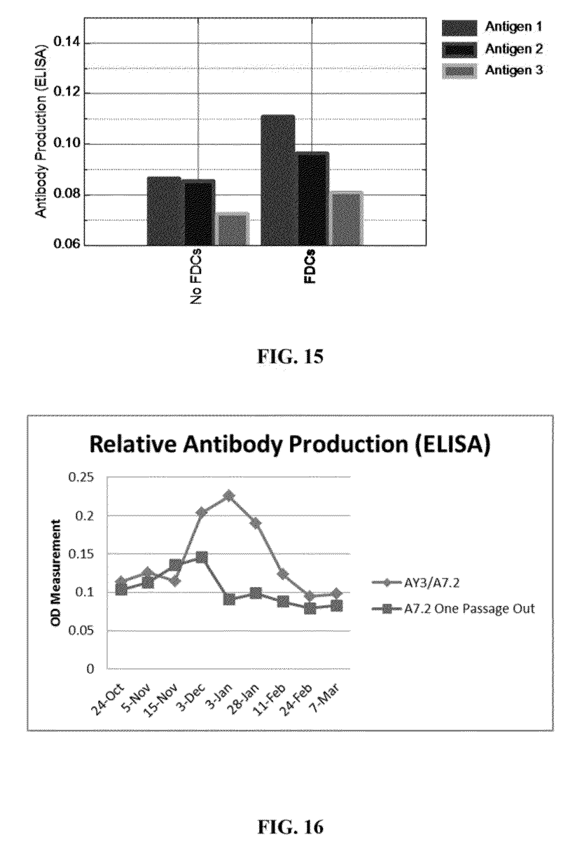

To test the ability of FDCs to promote Ab production of hybridomas in vitro, hybridoma/FDC co-cultures were monitored by ELISA over a 5 month period. Hybridoma supernatant was tested against several different antigens to determine Ab titer for each antigen (Ag). Results indicated that hybridomas in the presence of FDCs had elevated Ab production to all Ags: increasing from less than 0.09 to about 0.11 for Ag 1 (VP1), from about 0.085 to about 0.095 for Ag 2 (VP3), and from about 0.07 to about 0.08 for Ag 3 (AEV vaccine), representing differences of 20%, 11% and 13%, respectively (FIG. 15). The supernatant used for ELISA was not purified; therefore low OD numbers were not surprising. In general, any OD value above 0.07 is considered to indicate antibody production against the specific antigen and OD values above 0.10 are considered to indicate good antibody production.

The ability of the FDC/hybridoma co-culture to maintain boosted Ab production was indicated by ELISA monitoring over a 5 month period. For at least 3 months, the FDCs enhanced the production of Ab, by up to 85%, but the effect decreased by the 5th month (FIG. 16). To identify if hybridomas could maintain boosted Ab production when removed from co-culture with FDCs, some of the A7.2 cells, previously co-culturing with the FDCs, were split into a separate flask without FDCs. Once removed from the FDCs, elevated Ab production was maintained for a short period (about 2 months) but eventually declined. The decline in Ab production of the non-co-cultured cells occurred while the FDC co-cultured hybridoma cells had an additional boost in Ab production, as we see a difference of about 0.14 for the January 3rd ELISA, representing a difference of 85.44%. These results clearly indicate that FDCs have a positive effect on mAb production. The removal of FDCs caused the hybridomas to slowly lose their Ab producing ability and they never reached the same level of Ab production as that attained by those hybridomas still with the FDCs, 0.23 attained by A7.2 with FDCs compared to 0.15 attained by A7.2 no longer in the presence of FDCs, representing a difference of 43.51% (FIG. 16 and FIG. 10).

### Undetected Ig Heavy Chain

To identify the class of Ab that the hybridomas were producing. Ab isotyping was performed using a commercial mouse mAb isotyping kit. Ab isotyping was performed using supernatant from hybridoma cells in the presence of FDCs and also from hybridoma cells removed from the FDCs. Surprisingly, in the hybridomas that were removed from the FDCs, no heavy chain Ig was detected, although the light chain (Lambda) remained the same (Table 7).

TABLE 7
Ab isotyping results (QD values at 450 nm).

| | AY3/A7.2 | A7.2 One Passage Out |
|---|---|---|
| IgG1 | 0.056 | 0.059 |
| IgG2a | 0.054 | 0.059 |
| IgG2b | 0.058 | 0.06 |
| IgG3 | 0.057 | 0.06 |
| IgA | 0.054 | 0.055 |
| IgM | 0.174 | 0.057 |
| Kappa | 0.056 | 0.058 |
| Lambda | 0.424 | 0.362 |

Because no heavy chain Ig was detected, it was thought that class switching may have occurred. To explore the potential for hybridomas to have undergone class switch recombination, PCR were used to monitor AID mRNA expression in the cells. AID expression is important for triggering class switch recombination and somatic hypermutation; therefore, if AID was being expressed by the hybridomas, there would be evidence that those events had occurred or were occurring.

A PCR assay was conducted to identify if there was AID expression in the hybridoma cells. GAPDH (housekeeping gene) expression was used as a control to determine if cDNA had been synthesized properly. Upon positive results for GAPDH, PCR was used to identify if AID mRNA was being expressed. Interestingly, the hybridomas no longer in the presence of FDCs and had the unknown heavy chain Ig, were expressing AID. Since AID expression triggers somatic hypermutation and class switch recombination, it would appear that hybridomas removed, from the FDCs had or were undergoing these events. These results actually would coincide with what naturally occurs in vivo since it is not until after B cells have interacted with FDCs that class switch recombination occurs.

These results suggest that FDCs may be used to help boost Ab production of hybridomas and sustain production over a long period of time. Observations of hybridoma and FDC co-cultures revealed that these cells interact in a way that is very similar to what is seen in vivo with B cells and FDCs. In the GC, B cells require direct contact with FDCs for progression of events that lead to the differentiation of B cells into either plasma, cells or memory B cells. The hybridomas acted in a similar way as FIG. 14 showed hybridoma cells associating with the FDCs. Also, the hybridoma cells in culture with FDCs needed to be passaged at sooner intervals than the hybridoma cells without FDCs. This would indicate that the FDCs were increasing the proliferation rate of the hybridoma cells.

Once the hybridoma cells were no longer in the presence of FDCs, it appeared that they were able to undergo class switch recombination and somatic hypermutation as AID mRNA was expressed in those cells. In previous studies, it was shown that expression of AID was sufficient to induce somatic hypermutation in hybridoma cells, which represent plasma cells that are beyond the developmental stage that perform somatic hypermutation. The fact that hybridomas can be induced to undergo high rates of somatic hypermutation with expression of AID might allow one to obtain sub-clones that produce high affinity mAbs and/or antibodies that are more specific.

The success of this technology provides a way to improve hybridoma cultures by not only boosting their antibody production but also allowing somatic hypermutation and class switch recombination to occur, thereby generating a more diverse and effective group of antibodies against a specific target Ag.

FDCs have been isolated from multiple species and were able to adapt these cells to culture where they grow and maintain the physical characteristics that are seen in vivo. The above results indicate that these FDC lines preserve their functional characteristics as B cell proliferation was supported, FDC cells generated activated/memory B cells, and said cells enhanced hybridoma Ab production. In addition, the molecular mechanisms that increase specificity, affinity and biological activity of antibodies occurred in the FDC cultured hybridoma cells.

One element that may help optimize the system would be the use of certain cytokines that are known to be involved in GC reactions in vivo. Many factors help trigger and induce processes within the GC.

In order to screen vaccines, the front end of the immune system may be added to the system. The germinal center reaction represents the very end stage of the natural humoral immune response. When a protein antigen enters the body, antigen presenting cells (APCs) are needed to engulf the foreign threat, chop it up into smaller peptides, nuarate to secondary lymphoid organs, and then present those peptides, via T cells. The T cells are then activated and wait for matching B cells to travel to the lymph node, where the initial interaction of B cells and T cells will begin, creating the GC. Therefore, it is necessary to add APCs to the system to allow for the presentation of antigens to T cells.

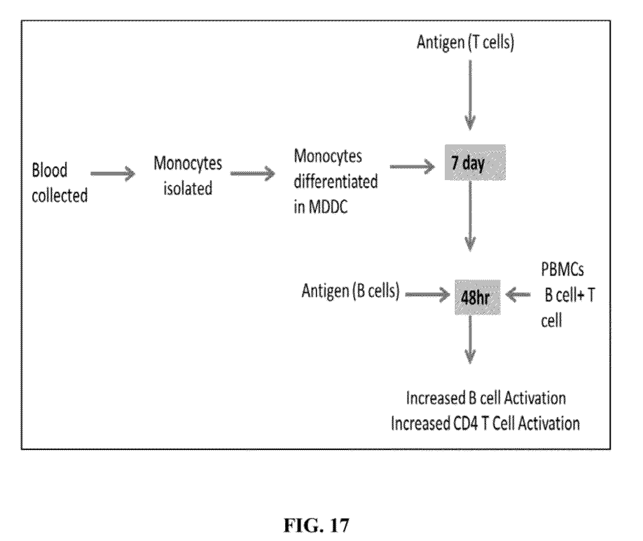

Successfully cultured monocyte derived dendritic cells (MDDCs), that can act as APCs in vitro, and have established their ability to engulf, process, and present antigen to T cells, which increased B cell and CD4 T cell activation have been established (FIG. 17). In embodiments, parts of the system are merged with APC components to mimic the entire humoral immune response.

Although the invention has been described with reference to the above examples, it will be understood that modifications and variations are encompassed within the spirit and scope of the invention. Accordingly, the invention is limited only by the following claims.

We claim herein:

1. A method of activating B-cells in vitro comprising:
contacting isolated follicular dendritic cells (FDC) from a subject immunized with an antigen and isolated peripheral blood mononuclear cells (PBMC) from a subject wherein said isolated PBMC comprise naïve B-cells;
incubating the contacted cells; and
detecting changes in the expression of a subset of cluster of differentiation (CD) antigens in the B-cell population;
wherein detection of changes in the expressed subset of CD antigens correlates with activation of naïve B-cells.

2. The method of claim 1, wherein B-cell activation comprises differentiation of said naïve B-cells to memory B-cells.

3. The method of claim 1, wherein the antigen is selected from the group consisting of Rift Valley Fever Virus (RVFV) subunits, Epizootic Hemorrhagic Disease Virus subunits, Influenza Virus subunits, ovalbumin, dextran, Transmissible Spongiform Encephalopathy prion proteins, and Alzheimer's disease mutated amyloid precursor protein (APP) and presenilins 1 and 2, SARS virus subunits, HIV subunits, Bovine Spongiform Encephalopathy prion proteins, SARS virus subunits, Avian Influenza Virus subunits, West Nile Virus subunits, Keyhole Limpet Hemocyanin, LPS, Bluetongue Virus subunits, Porcine Epidemic Diarrhea Virus (PEDV) subunits, and combinations thereof.

4. The method of claim 3, wherein the antigen is an RVFV subunit, comprising the amino acid sequence as set forth in SEQ ID NO: 1.

5. The method of claim 1, wherein the subject is a mammal.

6. The method of claim 1, further comprising contacting antigen presenting cells (APC) and T-cells with naïve B-cells prior to contacting naïve cells with FDC.

7. The method of claim 1, wherein the subset of CD antigens are selected from the group consisting of CD6, CD9, CD10, CD11b, CD11c, CD19, CD20, CD21, CD22, CD23, CD24, CD25, Cd26, CD27, CD28, CD30, CD32, CD35, CD37, CD38, CD39, CD40, CD45RO, CD45RA, CD45RB, CD49b, CD49c. CD49d, CD50, CD52, CD57, CD62L, CD69, CD70, CD72, CD73, CD74, CD75, CD76, CD79α, β, CD80, CD83, CDw84, CD85, CD86,CD89, CD97, CD98, CD119, CDw121b, CD122, CD124, CD125, CD126, CD127, CD130, CD132, CD135, CDw137, CD138, CD139 and combinations thereof.

8. The method of claim 1, wherein the change is reduction in B-cells expressing CD19 antigen.

9. The method of claim 1, wherein the change is an increase in B-cells expressing CD11b antigen, CD11c antigen or both CD11b antigen and CD11c antigen.

10. The method of claim 1, wherein said FDC supports proliferation of B-cells in vitro.

11. A method of promoting antibody production in hybridoma cells comprising:
fusing a melanoma cell with splenic B-cells of a subject immunized with an antigen;
cloning the resulting fused cells;
contacting isolated follicular dendritic cells (FDC) from a subject and the cloned fused cells and optionally removing said FDC from said cloned fused cells; and
determining the amount of antibody produced between fused cells contacted with FDC and non-contacted fused cells,
wherein ELISA OD values between about 0.07 to about 0.1 indicate antibody production against the antigen.

12. The method of claim 11, wherein subsequent removal of the FDC results in decline of the antibody production from the contacted fused cells.

13. The method of claim 12, wherein the contacted fused cells subsequent to FDC removal express activation-induced cytidine deaminase (AID).

14. The method of claim 13, wherein the contacted used cells subsequent to FDC removal undergo somatic hypermutation, class switch recombination or a combination thereof.

15. The method of claim 11, wherein the antigen is selected from the group consisting of Rift Valley Fever Virus (RVFV) subunits, Epizootic Hemorrhagic Disease Virus subunits, Influenza Virus subunits, ovalbumin, dextran, Transmissible Spongiform Encephalopathy prion proteins, and Alzheimer's disease mutated amyloid precursor protein (APP) and presenilins 1 and 2, SARS virus subunits, HIV subunits, Bovine Spongiform Encephalopathy prion proteins, SARS virus subunits, Avian Influenza Virus subunits, West Nile Virus subunits, Keyhole Limpet Hemocyanin, LPS, Bluetongue Virus subunits, Porcine Epidemic Diarrhea Virus (PEDV) subunits, and combinations thereof.

16. The method of claim 15, wherein the antigen is an RVFV subunit, comprising the amino acid sequence as set forth in SEQ ID NO:1.

17. An isolated, activated B-cell produced by the method of claim 1.

18. An isolated hybridoma cell produced by the method of claim 11 subsequent to contacting said FDC.

19. A FDC cell line AY3 having ______ accession no. ______.

20. An immunogenic agent comprising an antibody produced by the method of claim 11, wherein said immunogenic agent is a vaccine.
